(19) United States
(12) Patent Application Publication
(10) Pub. No.: US 2017/0142920 A1
(43) Pub. Date: May 25, 2017

(54) MATERIALS AND METHODS FOR LARGE SCALE PLANT GRAFTING

(71) Applicant: Beekenkamp Plants B. V., Maasdijk (NL)

(72) Inventor: Annie Cornelia Beekenkamp, Maasdijk (NL)

(21) Appl. No.: 14/946,090

(22) Filed: Nov. 19, 2015

(51) Int. Cl. A01H 5/06; A01G 17/02; A01G 17/00; A01H 5/08; A01G 1/06

(52) U.S. Cl. CPC A01H 5/06; A01H 5/08; A01G 1/06; A01G 17/005; A01G 17/02

(57) ABSTRACT

Embodiments of the present disclosure generally relate to materials and methods for plant grafting. In certain embodiments, the present disclosure provides materials and methods for efficient large scale grafting of potato rootstock with scion from the Solanaceae family. Certain embodiments involve grafting methods that significantly increase the yield of grafted plants produced. Given the commercial, nutritional, and horticultural advantages of grafted plants, embodiments of the present disclosure address the need for the development of improved methods for producing grafted plants and grafted plant products.

### FIELD

Embodiments of the present disclosure generally relate to materials and methods for plant grafting. In certain embodiments, as disclosed herein, materials and methods can provide for efficient large scale grafting of potato rootstock with scion from the Solanaceae family. Certain embodiments of the present disclosure also concern grafting methods that significantly increase yield of grafted plants produced compared to other methods.

### BACKGROUND

For centuries, plant grafting has been used in agriculture to enhance the health, yield, and fruit quality of various plant species, including, for example, fruit trees and grape vines. Large scale production of grafted vegetables emerged in Asia, where land has been intensively cultivated for years. For example, around the 1920s, Asian growers determined that grafting scion from watermelon plants onto squash or gourd rootstock significantly reduced the incidence of fusarium wilt (a fungal disease). Currently, at least 80% of Korean vegetables and at least 50% of all Japanese vegetables (including 95% of Japan's watermelons, oriental melons, greenhouse cucumbers, tomatoes and eggplants) are produced from grafted plants. In the United States, plant grafting is commonly used for various species of melons and tomatoes. Vegetable grafting is also used throughout Europe, including in Greece, Spain, France, Italy, and Morocco.

There are many advantages of grafted vegetables including, but not limited to, enhanced plant vigor, better disease resistance, improved tolerance to environmental stresses, and heavier crops that are produced over an extended harvest period. In one example, after success with grafting melons, Asian growers experimented with grafting tomato plants as a strategy to avoid soil-borne diseases like bacterial wilt, which can be hard to eradicate in a tomato crop because of its wide range of hosts and ability to persist for years in the soil. Plant grafting may also help plants ward off other infestations, including early blight (Alternaria solani), late blight (Phytophthora infestans), and blossom end-rot (a physiological disorder caused by low calcium levels). Grafted plants can also be more tolerant of environmental stresses like salinity or temperature extremes. The ability to withstand hotter and cooler temperatures can extend the growing season.

Additionally, even for those growers and gardeners fortunate enough to have fresh soil and ideal growing conditions, grafting can provide additional advantages. The vigorous rootstock increases the uptake of water and nutrients, for healthier and more beautiful plants having greater harvests without using chemical pesticides or fertilizers. Overall, grafted plants tend to produce larger harvests of better quality fruits over a longer period with fewer harmful inputs. However, it has been challenging for the horticultural industry to develop plant grafting methods that integrate the advantages of existing plant grafting techniques with the scale up potential of modern commercial agriculture.

### SUMMARY

Embodiments of the present disclosure can include a method for producing a grafted plant and grafted plant products. In accordance with these embodiments, the method includes grading cultured rootstock tissue to obtain at least one rootstock having a stem with a graft-compatible diameter, and making an angled cut through the at least one rootstock stem having a graft-compatible diameter and placing a stabilization device adjacent to the angled cut on the rootstock stem. The method can also include grading scion plants to obtain at least one scion having a stem with a graft-compatible diameter, making an angled cut through the at least one scion stem having a graft-compatible diameter, the angled cut substantially similar to the angled cut on the rootstock stem, and inserting the at least one cut scion stem into the stabilization device such that vascular tissue within the at least one cut scion stem substantially aligns with vascular tissue within the cut rootstock stem. The alignment of the vascular tissue of the at least one cut scion stem and the vascular tissue of the at least one cut rootstock stem can lead to inosculation of the vascular tissue and to the formation of at least one grafted plant.

In some embodiments of the present disclosure, the method can include the use of cultured rootstock tissue from the Solanaceae family, including Solanum tuberosum tissue. In certain embodiments, methods disclosed herein can include the use of scion from plants of the Solanaceae family, including one or more of Solanum melongena (eggplant), Solanum petunia, Solanum calibrachoa and Solanum lycopersicum (tomato). Embodiments of the method can also include the use of scion from the Capsicum genus. In certain embodiments, the rootstock of the grafted plant can be Solanum tuberosum and the scion of the grafted plant can be Solanum lycopersicum.

In accordance with these embodiments, the method also includes rootstock stems and scion stems having graft-compatible diameters from about 1.0 mm to about 2.0 mm. Embodiments of the method include the use of a silicone graft clip as a stabilization device, including a slot for a stabilization stake.

Some embodiments disclosed herein can include methods for transferring cultured rootstock into individual growth containers and maintaining the rootstock at temperatures from about 20° C. to about 25° C. and at light intensities from about 4000 lux to about 8000 lux for about 3 to about 8 days prior to grading. Embodiments also include making the angled cut through the at least one rootstock stem at temperatures from about 16° C. to about 20° C. and at light intensities from about 4000 lux to about 8000 lux about 9 days to about 14 days after grading. Embodiments also include sowing the seeds of the scion plants at temperatures from about 20° C. to about 25° C. and at light intensities from about 4000 lux to about 8000 lux about 5 days to about 10 days prior to grading. Embodiments also include making the angled cut through the at least one scion stem at temperatures from about 18° C. to about 22° C. and at light intensities from about 4000 lux to about 8000 lux about 5 days to about 10 days after grading.

In certain embodiments, the method can include inserting at least one cut scion stem into the stabilization device such that vascular tissue within the cut scion stem substantially aligns with vascular tissue within the cut rootstock stem. In some embodiments, this portion of the grafting process occurs at temperatures from about 20° C. to about 25° C. and at light intensities from about 4000 lux to about 8000 lux. Embodiments also include attaching a stabilization stake to the stabilization device about 6 days to about 8 days after inserting the at least one cut scion stem into the stabilization device such that vascular tissue within the at least one cut scion stem substantially aligns with vascular tissue within the cut rootstock stem. Embodiments also include grading the at least one grafted plant to determine overall viability about 6 days to about 9 days after inserting the at least one cut scion stem into the stabilization device such that vascular tissue within the at least one cut scion stem substantially aligns with vascular tissue within the cut rootstock stem.

In accordance with embodiments of the present disclosure, the yield of grafted plants can be at least 80% when the method is applied to a plurality of rootstock and scion. Embodiments of the method also include a grafted plant product produced by the rootstock and/or the scion of the at least one grafted plant. Yields of the grafted plant product produced by the rootstock and/or the scion of the at least one grafted plant can be from about 1.5 times to about 5.0 times greater than a non-grafted plant of the same species as the rootstock and/or the scion. Additionally, one or more organoleptic properties of the grafted plant product produced by the rootstock and/or the scion of the at least one grafted plant can be enhanced using the methods of the present disclosure, as compared to a non-grafted plant of the same species as the rootstock and/or the scion. Embodiments of the method also include grafted plants having enhanced disease resistance compared to non-grafted plants of the same species as either the rootstock or the scion.

As used herein, the terms “graft,” “grafting,” “engraft,” or “graftage” generally refer to a horticultural technique whereby the vascular tissue from one plant fuses with the vascular tissue of another plant, such that the two plants form a single grafted plant through the inosculation of their vascular tissue.

As used herein, the terms “scion” or “cion” generally refer to upper portion of a grafted plant that imparts the leaves, flowers, and/or fruit to the grafted plant. The scion generally contains the desired genetic material that will be propagated by the grafted plant.

As used herein, the terms “rootstock” or “stock” generally refer to the lower portion of a grafted plant that imparts the roots to the grafted plant. The rootstock can be used to improve the stress tolerance and disease resistance of the scion, among other advantages.

As used herein, the terms “grade” or “grading” generally refer to a process of assessing or evaluating plants or plant tissue using certain criteria or to identify certain attributes. For example, uniformity and synchronization of growth are important aspects of plant grafting, and both scion and rootstock can be graded to optimize the yield of grafted plants produced according to certain specified criteria.

The terms “determine,” “calculate,” and “compute,” and variations thereof, as used herein, are used interchangeably and include any type of methodology, process, mathematical operation or technique.

It is to be noted that the term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

Unless otherwise specified, any use of any form of the terms “connect,” “engage,” “couple,” “attach,” or any other term describing an interaction between elements is not meant to limit the interaction to direct interaction between the elements and may also include indirect interaction between the elements described.

In the following discussion and in the claims, the terms “including” and “comprising” are used in an open-ended fashion, and thus should be interpreted to mean “including, but not limited to . . . ”. The various characteristics mentioned above, as well as other features and characteristics described in more detail herein will be readily apparent to those skilled in the art with the aid of the present disclosure upon reading the following detailed description of the embodiments.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “means” as used herein shall be given its broadest possible interpretation in accordance with 35 U.S.C. §112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. The drawings simply illustrate preferred and alternative examples of how the disclosure can be made and used and are not to be construed as limiting the disclosure to the illustrated and described examples. Further features and advantages will become apparent from the following, more detailed, description of the various aspects, embodiments, and configurations of the disclosure, as illustrated by the drawings referenced below.

FIG. 1 is a representative diagram of a plant grafting timeline, according to one embodiment of the present disclosure.

FIG. 2A is a representative image of a cross-sectional view of a rootstock or scion stem, while FIG. 2B is a representative image of a grafted plant with a stabilization device adjacent to the grafting site, according to one embodiment of the present disclosure.

FIG. 3A is a representative image of cultured rootstock tissue from the Solanum family, while FIG. 3B is a representative image of seedlings of Solanum lycopersicum, according to one embodiment of the present disclosure.

FIG. 4 is a representative image of a grafted plant having grafted plant products produced by the scion (e.g., tomatoes) and the rootstock (e.g., potatoes), according to one embodiment of the present disclosure.

### DETAILED DESCRIPTION

Embodiments of the present disclosure generally relate to materials and methods for plant grafting. In certain embodiments, materials and methods of the present disclosure can provide for efficient large scale grafting of potato rootstock with scion from the Solanaceae family. Some embodiments of the present disclosure concern grafting methods that significantly increase the yield of grafted plants produced compared to other known methods.

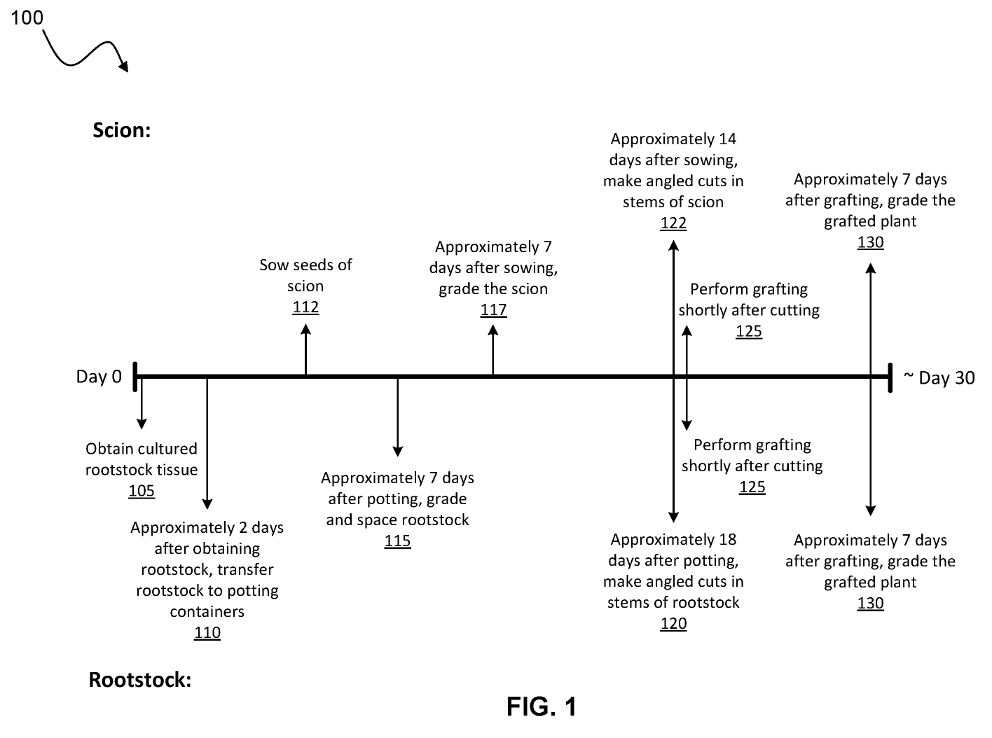

As illustrated in FIG. 1, the plant grafting methods of the present disclosure can be performed, for example, according to a plant grafting timeline 100. In certain embodiments, the plant grafting timeline 100 commences when cultured rootstock tissue is obtained 105 (approximately day 0). Approximately 2 days after the rootstock tissue is obtained 105, the rootstock can be transferred to potting containers such that each rootstock occupies a single potting container 110. In some embodiments, the rootstock can be transferred to potting containers from about 1 to about 3 days after the cultured rootstock tissue is obtained 105 (e.g., from about 3 to about 8 days prior to grading). In some embodiments, transferring the rootstock to individual potting containers 110 is performed at temperatures from about 20° C. to about 25° C. and at light intensities from about 4000 lux to about 8000 lux. In some embodiments, transferring the rootstock to individual containers 110 is performed at about 23° C. and at about 6000 lux.

Continuing with FIG. 1, the rootstock can be graded approximately 5-10 days after being transferred into individual potting containers 115. In some embodiments, the root stock is graded approximately 7 days after potting. Grading and spacing the rootstock can be done using various methods, as one of ordinary skill in the art would readily recognize based on the present disclosure. Generally, grading is used to assess and/or evaluate plants or plant tissue using certain criteria or to identify certain attributes. For example, uniformity and synchronization of growth are important aspects of plant grafting, and both scion and rootstock can be graded to optimize the yield of grafted plants produced according to these criteria. In some embodiments, grading involves identifying rootstock with stems having graft-compatible diameters. Graft-compatible stem diameters can range from about 0.5 mm to about 3.0 mm. In some embodiments, graft-compatible stem diameters can range from about 0.5 mm to about 3.0 mm, from about 0.5 mm to about 2.5 mm, from about 0.5 mm to about 2.0 mm, from about 1.0 mm to about 3.0 mm, from about 1.0 mm to about 2.5 mm, and from about 1.0 mm to about 2.0 mm. In some embodiments, graft-compatible diameters can range from about 1.5 mm to about 2.5 mm, from about 1.5 mm to about 2.0 mm, from about 1.5 mm to about 1.8 mm, and from about 1.2 mm to about 1.5 mm. In some embodiments, the rootstock is returned to a temperature of about 23° C. and light intensity of about 6000 lux after grading.

Approximately 9-14 days after grading, angled cuts are made through the stems of the rootstock 120 (e.g., approximately 16-21 days after the rootstock have been transferred into potting containers). In some embodiments, the angled cut is made through the stems of the rootstock 120 approximately 11 days after grading (e.g., approximately 18 days after the rootstock have been transferred into potting containers). In some embodiments, making an angled cut through the rootstock stem 120 is performed at temperatures from about 20° C. to about 25° C. and at light intensities from about 4000 lux to about 8000 lux. In some embodiments, making an angled cut through the rootstock stem 120 is performed at about 23° C. and at about 6000 lux.

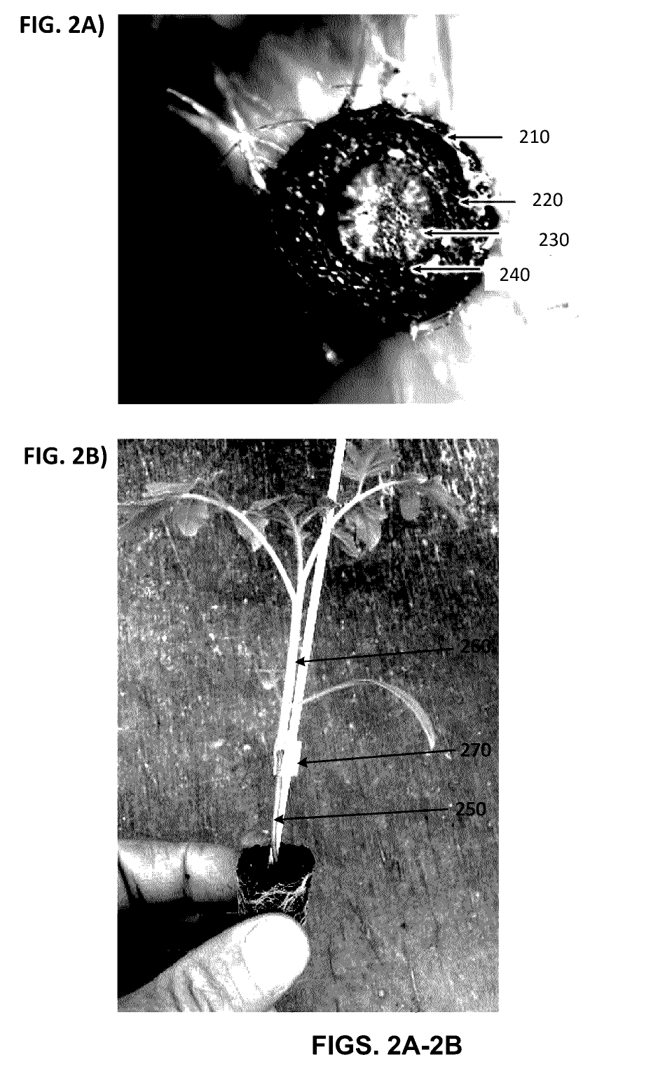

The angled cut made through the rootstock can expose the vascular tissue of the rootstock stem in preparation for grafting with the scion. As illustrated in FIG. 2A, vascular tissue in the stem of rootstock and scion generally comprises cortex 210, phloem, 220, vascular bundles within the xylem 230, and cambium 240, as would be readily recognized by one or ordinary skill in the art based on the present disclosure. As illustrated in FIG. 2B, for plant grafting to be successful, the vascular tissues of both the rootstock 250 and scion 260 should be substantially aligned in order for the vascular tissue to fuse, often referred to as inosculation. Successful plant grafting depends on a number of variables, including but not limited to, synchronizing the growth of the rootstock 250 and the scion 260 such that their stems reach graft-compatible diameters at approximately the same developmental time, thus greatly increasing the potential for successful grafting.

In some embodiments, a stabilization device 270 is placed on the rootstock stem adjacent to the site of the angled cut, as illustrated in FIG. 2B. Various stabilization devices can be used, including but not limited to, tape, plastic wrap, rubber bands, clips, and the like, or any combinations thereof. In some embodiments, the stabilization device 270 is a silicone clip that encompasses the site of the graft. In some embodiments, the silicone clip can be approximately from 1 cm to about 2 cm long and accommodate stems stem diameters from about 1.0 mm to about 2.0 mm. In some embodiments, the silicone clips accommodate stem diameters from about 1.2 mm to about 1.8 mm. In other embodiments, the silicone clips accommodate stem diameters from about 1.2 mm to about 1.5 mm. In some embodiments, the stabilization device 270 can further comprise a slot for the insertion of a stabilization stake, which supports the vertical position of the grafted plant and promotes proper growth.

Returning to the grafting timeline 100 of FIG. 1, embodiments of the present disclosure include the preparation of scion for grafting to rootstock. In some embodiments, seeds of the scion are sown 112 approximately 4-8 days after obtaining the cultured rootstock tissue 105 (e.g., 5-10 days prior to grading the scion). In some embodiments, the seeds of the scion are sown 112 approximately 6 days after obtaining the cultured rootstock tissue 105. Sowing the seeds of the scion 112 can be performed at temperatures from about 20° C. to about 25° C. and at light intensities from about 4000 lux to about 8000 lux. In some embodiments, sowing the seeds of the scion 112 is performed at about 23° C. and at about 6000 lux.

Approximately 5-10 days after sowing the seeds of the scion 112, the scion are graded 117. In some embodiments, the scion is graded 117 approximately 7 days after sowing the seeds of the scion 112. Grading the scion can be done using various methods, as one of ordinary skill in the art would readily recognize based on the present disclosure. Generally, grading is used to assess and/or evaluate plants or plant tissue using certain criteria or to identify certain attributes. For example, uniformity and synchronization of growth are important aspects of plant grafting, and both scion and rootstock can be graded to optimize the yield of grafted plants produced according to these criteria. In some embodiments, grading involves identifying scion with stems having graft-compatible diameters. Graft-compatible stem diameters can range from about 1.0 mm to about 2.0 mm. In some embodiments, graft-compatible stem diameters can range from about 1.2 mm to about 1.8 mm. In other embodiments, graft-compatible stem diameters can range from about 1.2 mm to about 1.5 mm. In some embodiments, the rootstock is returned to a temperature of from about 18° C. to about 22° C. and light intensity of about 6000 lux after grading.

Approximately 5-10 days after grading, angled cuts are made through the stems of the scion 122 (e.g., approximately 11-16 days after the seeds of the scion have been sown). In some embodiments, the angled cut is made through the stems of the scion 122 approximately 7 days after grading (e.g., approximately 14 days after the seeds of the scion have been sown). In some embodiments, making an angled cut through the scion stem 122 is performed at temperatures from about 20° C. to about 25° C. and at light intensities from about 4000 lux to about 8000 lux. In some embodiments, making an angled cut through the scion stem 122 is performed at about 23° C. and at about 6000 lux. The angled cut made through the scion can expose the vascular tissue of the scion stem in preparation of grafting with the rootstock, as described above. For plant grafting to be successful, the vascular tissues of both the rootstock and scion should be substantially aligned in order for the vascular tissue to fuse, often referred to as inosculation. Successful plant grafting depends on a number of variables, including but not limited to, synchronizing the growth of the rootstock and the scion such that their stems reach graft-compatible diameters at approximately the same developmental time, thus greatly increasing the potential for successful grafting.

As described above a stabilization device can be used to facilitate the alignment of the rootstock stem with the scion stem. In some embodiments, after the stabilization device has been placed on the cut stem of the rootstock, the cut stem of the scion can be inserted into the stabilization device. This initiates the grafting process 125, and is generally performed shortly after both the rootstock and scion have been cut. In some embodiments, inserting the cut stem into the stabilization device such that its vascular tissue substantially aligns with the vascular tissue of the cut stem of the rootstock (e.g., grafting 125) is performed at temperatures from about 20° C. to about 25° C. and at light intensities from about 4000 lux to about 8000 lux. In some embodiments, inserting the cut stem into the stabilization device such that its vascular tissue substantially aligns with the vascular tissue of the cut stem of the rootstock is performed at about 23° C. and at about 6000 lux. In some embodiments, a stabilization stake is affixed to the stabilization device to support the vertical position of the grafted plant and promote proper growth.

Approximately 6-9 days after inserting the cut scion stem into the stabilization device such that its vascular tissue substantially aligns with vascular tissue of the cut rootstock stem, the grafted rootstock/scion are graded 130. In some embodiments, the grafted rootstock/scion are graded approximately 7 days after inserting the cut scion stem into the stabilization device such that its vascular tissue substantially aligns with vascular tissue of the cut rootstock stem. Grading the grafted rootstock/scion can be performed using various methods, as one of ordinary skill in the art would readily recognize based on the present disclosure. Generally, the grafted rootstock/scion plants are hardened off shortly after grading at about 10° C.

The plant grafting methods of the present disclosure can be used with any suitable combinations of rootstock and scion, as would be readily recognized by one of ordinary skill in the art based on the present disclosure. In accordance with the embodiments of the present disclosure, rootstock can be any plants or plant tissue from the Solanaceae family (nightshades), and any varieties and/or derivatives thereof. In some embodiments, rootstock used with the plant grafting methods disclosed herein can include, but is not limited to, Solanum tuberosum plants or Solanum tuberosum plant tissue (also referred to as potato plants or potato tissue).

In some embodiments, rootstock can be a plant or seedling that has been potted in a suitable container. In other embodiments, rootstock can be from cultured rootstock tissue. For example, rootstock used with the plant grafting methods of the present disclosure can include cultured tissue from S. tuberosum obtained using plant tissue culture methods. Plant tissue culture involves maintaining and growing plant cells, tissues or organs, especially on artificial medium in suitable containers under controlled environmental conditions. The portion of the plant which is cultured is generally referred to as the explant, which includes any portion of a plant removed and grown in a test tube under sterile conditions in nutrient media. The capacity to generate a whole plant from an explant is called cellular totipotency.

In some embodiments of the present disclosure, S. tuberosum tissue is cultured using micropropagation. Micropropagation is a tissue culture technique used for rapid vegetative multiplication by using small propagules (“micro”). Micropropagation produces plants that are genetically identical to the original plant from which the propagule was removed. The genetically identical plants developed from any part of a plant by tissue culture/micropropagation are generally referred to as somaclones. Advantages of micropropagation include, but are not limited to, the rapid and efficient multiplication of genetically identical plants, the ability to grow plants independent of seasonal conditions, and the ability to synchronize the growth of the plants such that their overall sizes are substantially similar. The synchronization of growth is especially advantageous for grafting applications, as described herein.

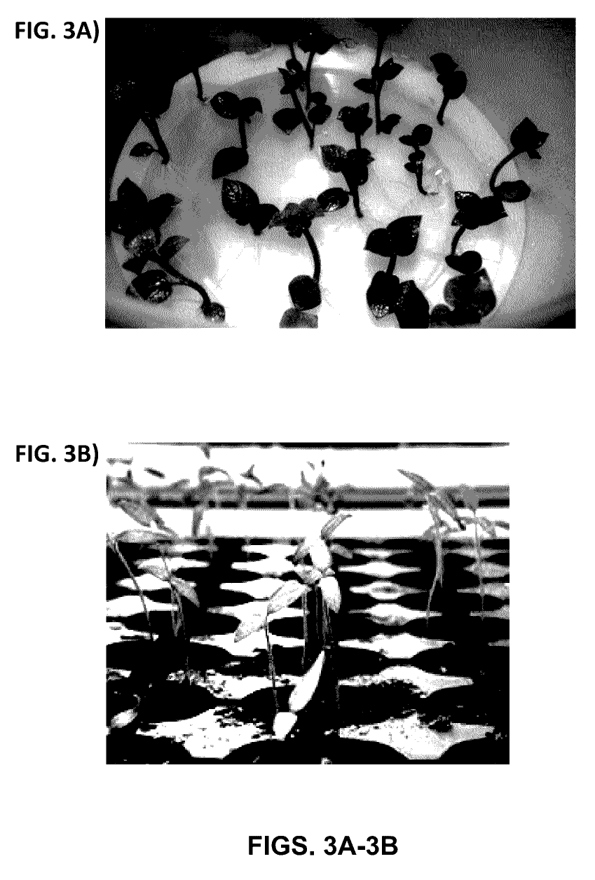

In some embodiments of the present disclosure, S. tuberosum tissue that is cultured using micropropagation can be used as rootstock for subsequent plant grafting procedures, as illustrated in FIG. 3A, for example. The ability to synchronize the growth of the cultured S. tuberosum rootstock tissue using micropropagation, for example, can optimize and/or maximize the yield of successfully grafted plants (e.g., maximize the production of plants having graft-compatible stem diameters and thus maximize the yield of successfully grafted plants).

Additionally, as one of ordinary skill in the art would readily recognize, the use of cultured rootstock tissue for grafting purposes can be technically challenging, and in some cases, the technical challenges can render plant grafting impossible or futile. For example, rootstock tissue is typically obtained from tubers or young plants that have been propagated with seeds (e.g., seedlings). Rootstock tissue obtained from these more traditional sources requires less skill, equipment and cost to establish and propagate, as compared to the equipment and cost required to obtain an equivalent amount cultured rootstock tissue. Sterile laboratory conditions and expensive laboratory equipment are required to obtain cultured rootstock tissue, whereas non-sterile, less expensive greenhouse systems (e.g., layered bed construction) are all that is required to obtain rootstock tissue from more traditional sources. Another disadvantage of using cultured rootstock tissue for plant grafting involves the fact that all plants propagated using tissue culture methods are from the same source of genetic material, making them all equally vulnerable to environmental stressors, like infections and pests. Therefore, there are many important variables that need to be considered when using cultured rootstock tissue for producing grafted plants.

In some embodiments of the present disclosure, scion can be any plants or plant tissue from the Solanaceae family, as well as any varieties and/or derivatives thereof. In some embodiments, scion used with the plant grafting methods of the present disclosure includes Solanum melongena (eggplant), Solanum petunia, Solanum calibrachoa, and Solanum lycopersicum (tomato). In other embodiments, scion used with the plant grafting methods of the present disclosure includes plants are from the Capsicum genus. In some embodiments, scion used with the plant grafting methods of the present disclosure includes S. lycopersicum. As illustrated in FIG. 3B, for example, seeds of S. lycopersicum plants can be grown in individual potting containers until the reaching a point of growth whereby their stems are graft-compatible with the stems of rootstock.

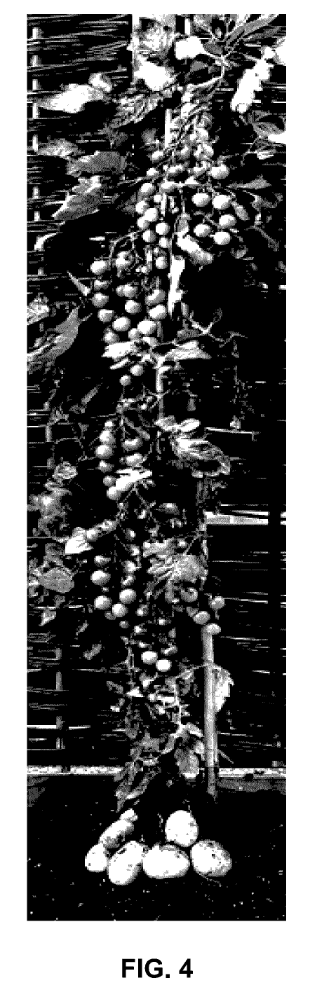

In some embodiments of the present disclosure, the rootstock of the grafted plant is S. tuberosum (potato) obtained from cultured tissue, and the scion of the grafted plant is S. lycopersicum (tomato), as illustrated in FIG. 4. Grafting S. tuberosum rootstock obtained from cultured tissue explants with S. lycopersicum scion can allow for the efficient scaling up of the plant grafting process and can increase the yield of successfully grafted plants due to the synchronization of the growth of the scion and rootstock to obtain graft-compatible stem diameters. In accordance with the embodiments of the present disclosure, the yield of grafted plants can be at least 80%. In some cases, the yield can be at least 81%, 82%, 83%, 84%, 85%, 86%, 87%, 88%, or at least 89%. In some cases, the yield can be at least 90%. In some cases, the yield can be at least 91%, 92%, 93%, 94%, 95%, 96%, 97%, 98%, or at least 99%.

In some embodiments, grafting S. tuberosum rootstock obtained from cultured tissue explants with S. lycopersicum scion confers various advantages to the grafted plants, as compared to non-grafted plants of the same or similar species. For example, grafting S. tuberosum rootstock obtained from cultured tissue explants with S. lycopersicum scion can confer enhanced disease resistance to the scion, as compared to a non-grafted plant of a similar species, including resistance to various soil-based pathogens. The ability to confer enhanced disease resistance is important for many varieties of S. lycopersicum scion (e.g., heirloom tomatoes).

In some embodiments, grafting S. tuberosum rootstock obtained from cultured tissue explants with S. lycopersicum scion enhances the yield of the various grafted plant products produced by the grafted plant. For example, grafting S. tuberosum rootstock obtained from cultured tissue explants with S. lycopersicum scion can increase the yield of fruit (e.g., tomatoes) from the S. lycopersicum scion from about 1.5 times to about 5.0 times, as compared to a non-grafted plant of a similar species. In some cases, the fruit yield is increased from about 1.5 times to about 4.5 times as compared to a non-grafted plant of a similar species. In some cases, the fruit yield is increased from about 1.5 times to about 4.0 times as compared to a non-grafted plant of a similar species. In some cases, the fruit yield is increased from about 1.5 times to about 3.5 times as compared to a non-grafted plant of a similar species. In some cases, the fruit yield is increased from about 1.5 times to about 3.0 times as compared to a non-grafted plant of a similar species. In some cases, the fruit yield is increased from about 1.5 times to about 2.5 times as compared to a non-grafted plant of a similar species. In some cases, the fruit yield is increased from about 1.5 times to about 2.0 times as compared to a non-grafted plant of a similar species.

In some embodiments, grafting S. tuberosum rootstock obtained from cultured tissue explants with S. lycopersicum scion enhances the yield of the grafted plant products produced by the rootstock. For example, grafting S. tuberosum rootstock obtained from cultured tissue explants with S. lycopersicum scion can increase the yield of potatoes produced from the rootstock from about 1.5 times to about 5.0 times, as compared to a non-grafted plant of a similar species. In some cases, the potato yield is increased from about 1.5 times to about 4.5 times as compared to a non-grafted plant of a similar species. In some cases, the potato yield is increased from about 1.5 times to about 4.0 times as compared to a non-grafted plant of a similar species. In some cases, the potato yield is increased from about 1.5 times to about 3.5 times as compared to a non-grafted plant of a similar species. In some cases, the potato yield is increased from about 1.5 times to about 3.0 times as compared to a non-grafted plant of a similar species. In some cases, the potato yield is increased from about 1.5 times to about 2.5 times as compared to a non-grafted plant of a similar species. In some cases, the potato yield is increased from about 1.5 times to about 2.0 times as compared to a non-grafted plant of a similar species.

In other embodiments, grafting S. tuberosum rootstock obtained from cultured tissue explants with S. lycopersicum scion enhances one or more organoleptic properties of the of the various grafted plant products produced by the grafted plant. For example, grafting S. tuberosum rootstock obtained from cultured tissue explants with S. lycopersicum scion can produce tomatoes and/or potatoes with enhanced organoleptic properties, as compared to non-grafted plants of similar species. As would be appreciated by one of ordinary skill in the art based on the present disclosure, various grafted plant products obtained using the methods disclose herein include, but are not limited to, potatoes, tomatoes, peppers, chili peppers, bell peppers, eggplant, and the like.

At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R₁, and an upper limit, Rᵤ, is disclosed, any number falling within the range is specifically disclosed. In particular, the following numbers within the range are specifically disclosed: R=R₁+k*(Rᵤ−R₁), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent.

Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present disclosure.

The present disclosure, in various aspects, embodiments, and configurations, includes components, methods, processes, systems and/or apparatus substantially as depicted and described herein, including various aspects, embodiments, configurations, sub combinations, and subsets thereof. Those of skill in the art will understand how to make and use the various aspects, aspects, embodiments, and configurations, after understanding the present disclosure. The present disclosure, in various aspects, embodiments, and configurations, includes providing compositions and processes in the absence of items not depicted and/or described herein or in various aspects, embodiments, and configurations hereof, including in the absence of such items as may have been used in previous compositions or processes, e.g., for improving performance, achieving ease and\or reducing cost of implementation.

### Examples

The following examples are included to illustrate various embodiments. It should be appreciated by those of skill in the art that the techniques disclosed in the examples that follow represent techniques discovered to function well in the practice of the claimed methods, compositions and apparatus. However, those of skill in the art should, in light of the present disclosure, appreciate that many changes may be made in the specific embodiments which are disclosed and still obtain a like or similar result without departing from the spirit and scope of the invention.

Generally, these data indicate that the use of cultured rootstock tissue can greatly enhance the percent yield of successfully grafted plants, as compared to the use of traditional sources of rootstock tissue.

The above examples, embodiments, definitions and explanations should not be taken as limiting the full metes and bounds of the invention. The present disclosure, in various aspects, embodiments, and configurations, includes components, methods, processes, systems and/or apparatus substantially as depicted and described herein, including various aspects, embodiments, configurations, sub combinations, and subsets thereof. Those of skill in the art will understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, e.g., for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A plant grafting method for producing a grafted plant, the method comprising:
grading cultured rootstock tissue to obtain at least one rootstock having a stem with a graft-compatible diameter;
making an angled cut through the at least one rootstock stem having a graft-compatible diameter and placing a stabilization device adjacent to the angled cut on the rootstock stem;
inserting at least one cut scion stem into the stabilization device such that vascular tissue within the at least one cut scion stem substantially aligns with vascular tissue within the cut rootstock stem;
wherein the vascular tissue of the at least one cut scion stem and the vascular tissue of the at least one cut rootstock stem inosculate to form the grafted plant.

2. The method of claim 1, further comprising:
grading scion plants to obtain the at least one scion, wherein the at least one scion has a stem with a graft-compatible diameter;
making an angled cut through the at least one scion stem having a graft-compatible diameter, the angled cut substantially similar to the angled cut on the rootstock stem

3. The method of claim 1, wherein the cultured rootstock tissue is from the Solanaceae family.

4. The method of claim 1, wherein the cultured rootstock tissue is Solanum tuberosum tissue.

5. The method of claim 1, wherein the scion plants are from the Solanaceae family.

6. The method of claim 1, wherein the scion plants comprise one or more of Solanum melongena (eggplant), Solanum petunia, Solanum calibrachoa, and Solanum lycopersicum (tomato).

7. The method of claim 1, wherein the scion plants comprise plants from the Capsicum genus.

8. The method of claim 1, wherein the rootstock of the grafted plant is Solanum tuberosum and the scion of the grafted plant is Solanum lycopersicum.

9. The method of claim 1, wherein the graft-compatible diameter of the at least one rootstock stem and the at least one scion stem is about 1.0 mm to about 2.0 mm.

10. The method of claim 1, wherein the stabilization device is a silicone graft clip with a slot for a stabilization stake.

11. The method of claim 1, wherein the cultured rootstock tissue is transferred into individual growth containers and maintained at temperatures from about 20° C. to about 25° C. and at light intensities from about 4000 lux to about 8000 lux for about 3 days to about 8 days prior to grading.

12. The method of claim 1, wherein making an angled cut through the at least one rootstock stem occurs at temperatures from about 16° C. to about 20° C. and at light intensities from about 4000 lux to about 8000 lux about 9 days to about 14 days after grading.

13. The method of claim 1, wherein making an angled cut through the at least one scion stem occurs at temperatures from about 18° C. to about 22° C. and at light intensities from about 4000 lux to about 8000 lux about 5 days to about 10 days after grading.

14. The method of claim 1, wherein inserting the at least one cut scion stem into the stabilization device such that vascular tissue within the at least one cut scion stem substantially aligns with vascular tissue within the cut rootstock stem occurs at temperatures from about 20° C. to about 25° C. and at light intensities from about 4000 lux to about 8000 lux.

15. The method of claim 1, further comprising attaching a stabilization stake to the stabilization device about 6 to about 8 days after inserting the at least one cut scion stem into the stabilization device such that vascular tissue within the at least one cut scion stem substantially aligns with vascular tissue within the cut rootstock stem.

16. The method of claim 1, further comprising grading the at least one grafted plant to determine overall viability about 6 to about 9 days after inserting the at least one cut scion stem into the stabilization device such that vascular tissue within the at least one cut scion stem substantially aligns with vascular tissue within the cut rootstock stem.

17. The method of claim 1, wherein the yield of grafted plants is at least 80% when the method is applied to a plurality of rootstock and scion.

18. A grafted plant produced using the method of claim 1.

19. The grafted plant of claim 18, wherein the cultured rootstock tissue is from the Solanaceae family.

20. The grafted plant of claim 18, wherein the cultured rootstock tissue is Solanum tuberosum tissue.

21. The grafted plant of claim 18, wherein the scion plants are from the Solanaceae family.

22. The grafted plant of claim 18, wherein the scion plants comprise one or more of Solanum melongena (eggplant), Solanum petunia, Solanum calibrachoa, and Solanum lycopersicum (tomato).

23. The grafted plant of claim 18, wherein the scion plants comprise plants from the Capsicum genus.

24. The grafted plant of claim 18, wherein the rootstock of the grafted plant is Solanum tuberosum and the scion of the grafted plant is Solanum lycopersicum.

25. A grafted plant product produced by the rootstock of the at least one grafted plant produced using the method of claim 1.

26. The grafted plant product of claim 18, wherein the yield of the grafted plant product produced by the rootstock of the at least one grafted plant is from about 1.5 times to about 5.0 times greater than a non-grafted plant of the same species as the rootstock.

27. A grafted plant product produced by the scion of the at least one grafted plant produced using the method of claim 1.

28. The grafted plant product of claim 21, wherein the yield of the grafted plant product produced by the scion of the at least one grafted plant is from about 1.5 times to about 5.0 times greater than a non-grafted plant of the same species as the scion.
